(19) United States
(12) Patent Application Publication
(10) Pub. No.: US 2014/0188196 A1
(43) Pub. Date: Jul. 3, 2014

(54) Methods For Weight Treatment in Animals Utilizing Narrow Spectrum Light

(71) Applicant: Michael Ryan, Castro Valley, CA (US)

(72) Inventor: Michael Ryan, Castro Valley, CA (US)

(21) Appl. No.: 14/191,399

(22) Filed: Feb. 26, 2014

Related U.S. Application Data
(63) Continuation of application No. 13/052,825, filed on Mar. 21, 2011, now abandoned.
(60) Provisional application No. 61/316,381, filed on Mar. 23, 2010.

(51) Int. Cl. A61N 5/06; A61D 99/00

(52) U.S. Cl. CPC A61N 5/0622; A61D 99/00
USPC 607/88

(57) ABSTRACT

Methods of weight treatment in animals are presented including: stimulating a retinal ganglia of the animal by an exposure to a narrow spectrum light. In some embodiments, the stimulating the retinal ganglia of the animal is associated with a stimulation of melanopsin. In some embodiments, the stimulation of melanopsin is associated with a inhibition of melatonin secretion. In some embodiments, inhibition of melatonin secretion is associated with a stimulation of leptin secretion. In some embodiments, the stimulation of leptin secretion is associated with a decrease in appetite in the animal. In some embodiments, the exposure to the narrow spectrum light is associated with a weight loss in the animal. In some embodiments, the narrow spectrum light is in a spectrum range of approximately 435 to 520 nanometers.

### BACKGROUND

As is typically well-known, animals experience changes in behavior patterns corresponding with seasonal changes. For example, in summer months, when weather is hot, mammals will often be less active during mid-day hours when temperatures are more extreme and more active during early morning, and late afternoon hours when temperatures are more moderate. In contrast, during winter months, when weather is generally colder, mammals will often be more active during mid-day hours when temperatures are more moderate. As such, a pet owner may expect that their animal may not respond positively to a fixed exercise schedule that ignores seasonal changes.

In addition, animals may experience physiological changes corresponding with seasonal changes. For example, in the months preceding summer, furry mammals such as cats and dogs may experience a decrease in coat density. Furthermore, mammals may experience weight loss as they become more active. These changes are likely an evolutionary response to warmer temperatures. In contrast, in the months preceding winter, these same mammals may experience an increase in coat density and coarseness. Likewise, these mammals may experience weight gains as their bodies store excess energy for lean winter months.

For a pet owner, the seasonal changes may cause some confusion when caring for their pet. Particularly for indoor pets, the evolutionary changes described may not be required for the animal's survival since the animal's movement is restricted to a climate controlled environment. In those cases, changes in the pet's behavior and physiology may be detrimental to the pets health and well-being. As such, methods for weight treatment in animals utilizing narrow spectrum light are presented herein.

### BRIEF SUMMARY

The following presents a simplified summary of some embodiments of the invention in order to provide a basic understanding of the invention. This summary is not an extensive overview of the invention. It is not intended to identify key/critical elements of the invention or to delineate the scope of the invention. Its sole purpose is to present some embodiments of the invention in a simplified form as a prelude to the more detailed description that is presented below.

As such, methods of weight treatment in animals are presented including: stimulating a retinal ganglia of the animal by an exposure to a narrow spectrum light. In some embodiments, the stimulating the retinal ganglia of the animal is associated with a stimulation of melanopsin. In some embodiments, the stimulation of melanopsin is associated with a inhibition of melatonin secretion. In some embodiments, the inhibition of melatonin secretion is associated with a stimulation of leptin secretion. In some embodiments, the stimulation of leptin secretion is associated with a decrease in appetite in the animal. In some embodiments, the exposure to the narrow spectrum light is associated with a weight loss in the animal. In some embodiments, the narrow spectrum light is in a spectrum range of approximately 435 to 520 nanometers. In some embodiments, the exposure to the narrow spectrum light is continuous. In some embodiments, the exposure to the narrow spectrum light is associated with an inhibited hibernation-like cycle in the animal. In some embodiments, the narrow spectrum light is provided by a source selected from the group consisting of: an incandescent bulb, a fluorescent bulb, and a plurality of light emitting diodes, and where the lamp has a wattage in a range of approximately 5 to 300 watts.

### BRIEF DESCRIPTION OF THE SEVERAL VIEWS OF THE DRAWINGS

The present invention is illustrated by way of example, and not by way of limitation, in the figures of the accompanying drawings and in which like reference numerals refer to similar elements and in which:

FIG. 1 is an illustrative representation of an orthogonal view of an animal treatment pad in accordance with embodiments of the present invention;

FIG. 2 is an illustrative representation of an orthogonal view of an animal treatment pad in use in accordance with embodiments of the present invention;

FIG. 3 is an illustrative representation of an orthogonal view of an animal treatment pad in use in accordance with embodiments of the present invention;

FIG. 4 is an illustrative schematic representation of a power control system in accordance with embodiments of the present invention;

FIG. 5 is an illustrative representation of user adjustable inputs in accordance with embodiments of the present invention;

FIG. 6 is an illustrative graph of an intensity curve for automatically adjusting light intensity in accordance with embodiments of the present invention; and

FIG. 7 is an illustrative representation of therapeutic effects in utilizing embodiments of the present invention.

### DETAILED DESCRIPTION

The present invention will now be described in detail with reference to a few embodiments thereof as illustrated in the accompanying drawings. In the following description, numerous specific details are set forth in order to provide a thorough understanding of the present invention. It will be apparent, however, to one skilled in the art, that the present invention may be practiced without some or all of these specific details. In other instances, well known process steps and/or structures have not been described in detail in order to not unnecessarily obscure the present invention.

Without being bound by theory, experimental use has demonstrated that use of embodiments provided herein have resulted in several beneficial aspects of pet care. For example, in utilizing embodiments provided herein, pets may be treated such that appetite increase during months preceding winter and during winter is avoided. As such, seasonal weight gain may, in some examples, be avoided. Further, use of embodiments provided herein has resulted in softer and silkier coats for pets, which characteristics are typically associated with summer months. Without being bound by theory, it is suggested that the use of a narrow spectrum light may function to trick an animal's evolutionary control system to respond to that is essentially a continuous summer. Experimental use has also resulted in a marked decrease in shedding and, therefore, a commensurate decrease in hairball production, which leads to an overall increase in general health of a pet. In addition, experimental use has further resulted in a marked decrease in vermin infestations such as flea infestation further leading to an overall increase in general health of a pet. In addition, since lamps, such as incandescent and fluorescent lamps emit heat, embodiments are defined in which intensity may be adjusted to avoid overheating a pet. However, in other embodiments, intensity may be adjusted to provide warmth to a pet.

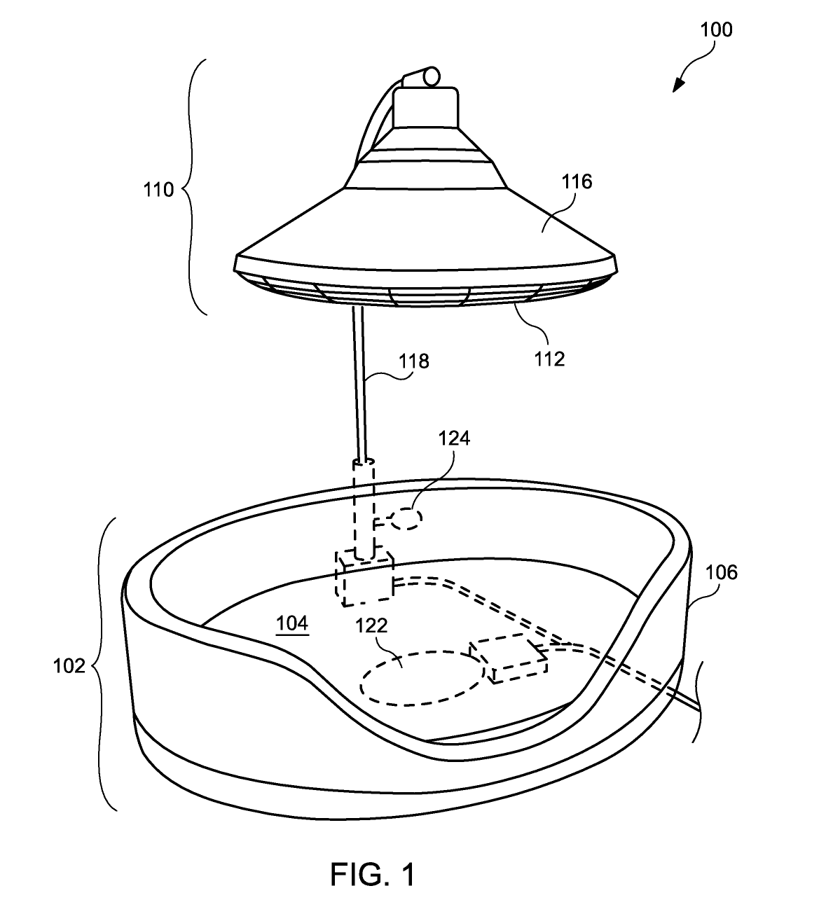

FIG. 1 is an illustrative representation of an orthogonal view of an animal treatment pad 100 in accordance with embodiments of the present invention. As illustrated, animal treatment pad 100 may include base 102. Base 102 may include padded area 104 to provide comfort to an animal utilizing embodiments described herein. In some embodiments, base 102 may include sidewall 106 disposed at least substantially perpendicular with base 102. In some embodiments, sidewall 106 is a removable sidewall. In other embodiments, sidewall 106 is a fixed sidewall. In colder environments, sidewall 106 may provide additional warmth for an animal. In some examples, it may be desirable to physically contain a small animal such as a newborn kitten. In those examples, sidewalls may provide at least some additional security for the animal. Lamp fixture 110 may be positioned to illuminate at least a portion of base 102. Adjusting lamp fixture 110 may be fixedly connected with base 102, or may be removably connected with base 102 via neck 118, which may be rigid or semi-rigid without limitation and without departing from embodiments herein. In embodiments, lamp fixture 110 may be vertically adjusted. Vertical adjustment may be utilized to accommodate differently sized animals as well as to adjust intensity of light reaching an animal.

Lamp fixture 110 may further include narrow spectrum lamp mounted in a lamp socket (not shown) for providing light treatment for animals. In some embodiments, a narrow spectrum lamp includes a wattage in a range of approximately 5 to 300 watts, more preferably 25 to 150 watts. In other embodiments, narrow spectrum lamp emits light in a spectrum range of approximately 520-435 nm. As noted above, experimental data has demonstrated without being bound by theory that treatment in this spectrum range has resulted in softer and silkier coats as well as appetite regulation in animals. Opaque lamp shade 116 may be utilized to limit or prevent light leakage to surrounding areas. In addition, lamp fixture 110 may further include protective screen 112 in some embodiments to prevent unintentional contact with the narrow spectrum lamp. In this manner, an animal may be safely treated without fear of inadvertent injury from heat generated by the narrow spectrum lamp.

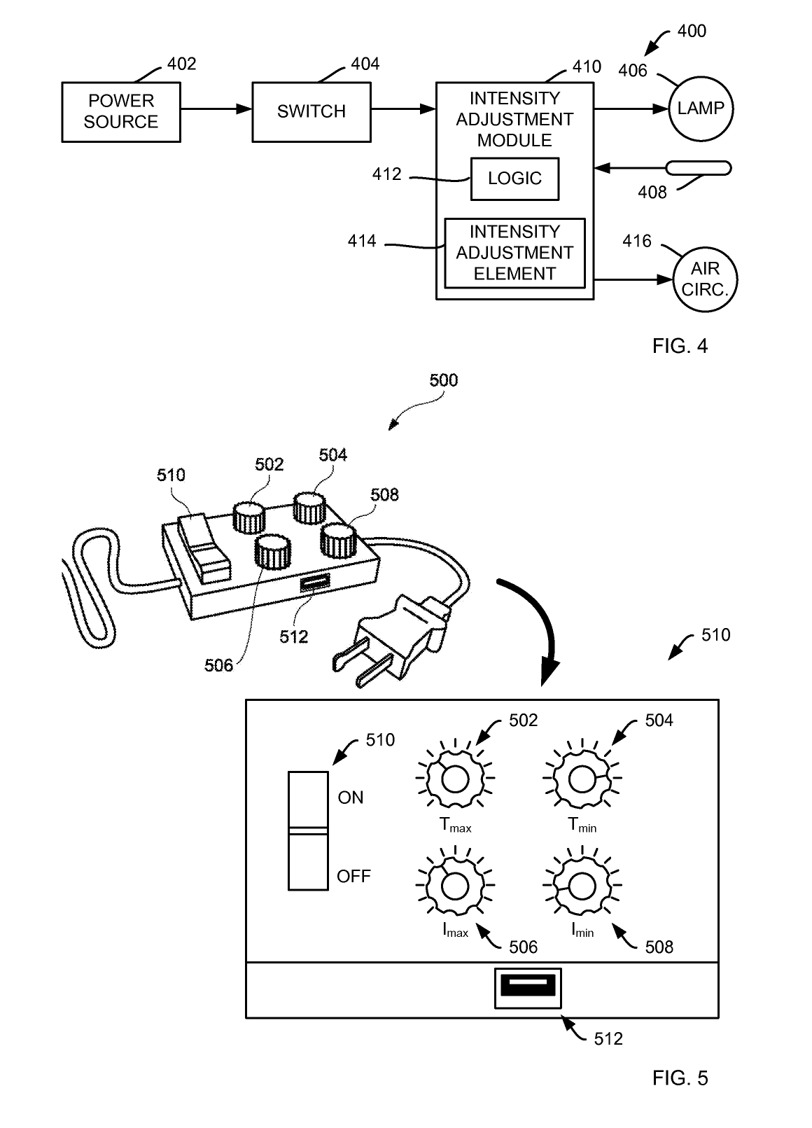

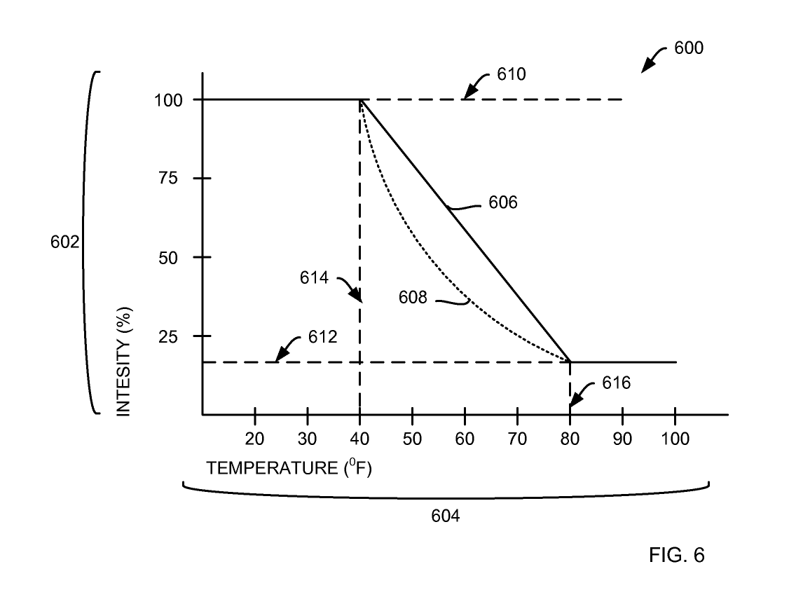

In some embodiments, a power control system may be utilized to adjust intensity of light in treating animals. User adjustable inputs may be utilized in coordination with pressure switch 122 and thermocouple 124. In some embodiments, pressure switch may be configured to turn on the device when an animal is on the pad. In this manner power savings may be realized as well as extended working life of narrow spectrum lamps. Any number of switches may be utilized including without limitation: a user activated switch, a pressure sensitive switch configured for automatically activating when an animal is an the pad, an inductive switch configured for automatically activating when an animal is detected, and a motion switch configured for automatically activating when motion is detected. User adjustable inputs are discussed in further detail below for FIGS. 4-6. In addition, utilization of pressure switches and thermocouples are discussed in further detail below for FIGS. 4 and 6. Animal treatment pad may be powered by a power source utilizing a power source connection (not shown). An power source known in the art may be utilized without departing from the present invention including without limitation: an alternating current power source, a direct current power source, and a solar cell power source.

In other embodiments, an air circulating device (not shown) may be utilized in coordination with embodiments disclosed herein. In some embodiments, an air circulating device may be vented through opaque lamp shade 116. In those embodiments, air circulating devices may be configured to both “push” and “pull” air through the shade. In this manner, heat from lamp fixture may either be directed toward an animal (i.e. push) or away from an animal (i.e. pull). In some embodiments, circulation devices may be attached with neck 118. Air circulating devices may be single speed, variable speed, or multi-speed in embodiments.

In still other embodiments, a weighing device (not shown) may be utilized in coordination with embodiments disclosed herein. In some embodiments, it may be desirable to track an animal's weight. This may be particularly useful in determining whether a treatment regime is effective. For example, if weight loss increases with an increase in light exposure, a user may elect to reduce light exposure to stabilize weight or reverse weight loss. In another example, weight tracking may provide feedback to an owner to initiate an increase or decrease in amount of food being given to an animal or pet. In some embodiments, weight may be tracked by logic element described in further detail below for FIG. 4.

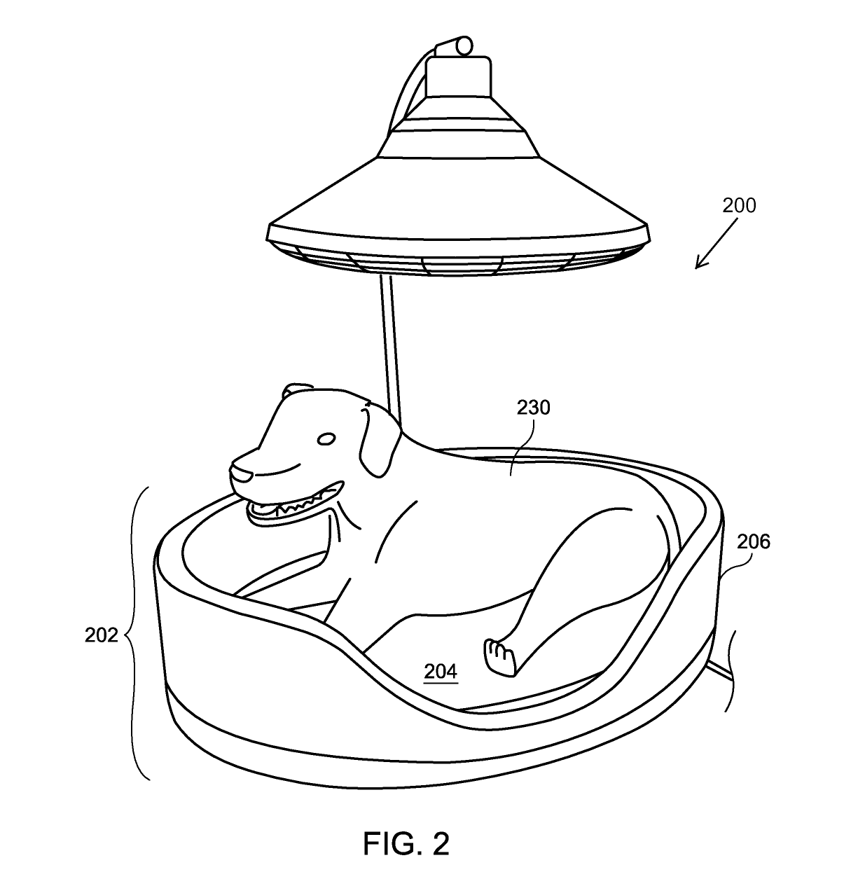

FIG. 2 is an illustrative representation of an orthogonal view of animal treatment pad 200 in use in accordance with embodiments of the present invention. In particular, the illustrated embodiment includes an animal 230 in treatment utilizing sidewall 206. In some embodiments, sidewall 206 is a removable sidewall. In other embodiments, sidewall 206 is a fixed sidewall. As illustrated, animal treatment pad 200 may include base 202. Base 202 may include padded area 204 to provide comfort to an animal utilizing embodiments described herein. In some embodiments, base 202 may include sidewall 206 disposed at least substantially perpendicular with base 202. In colder environments, sidewall 206 may provide additional warmth for an animal. In some examples, it may be desirable to physically contain a small animal such as a newborn kitten. In those examples, sidewalls may provide at least some additional security for the animal. As illustrated, sidewall 206 includes a side opening for ease of entry and egress of the animal. However, sidewalls may be configured with no side opening without departing from the present invention.

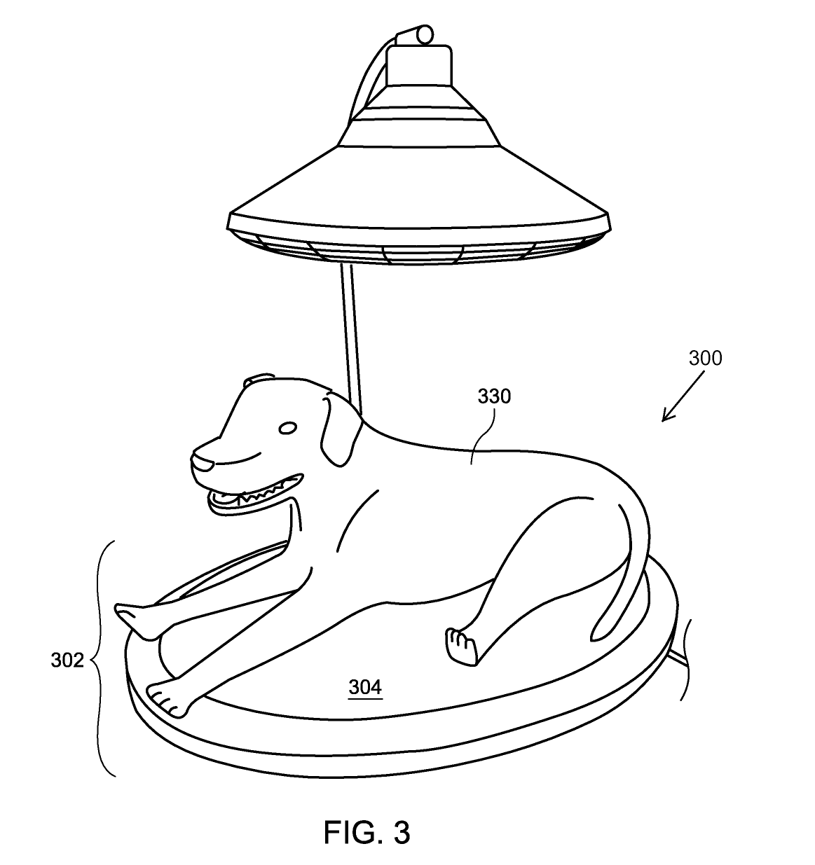

FIG. 3 is an illustrative representation of an orthogonal view of an animal treatment pad 300 in use in accordance with embodiments of the present invention. In particular, the illustrated embodiment includes an animal 330 in treatment without a sidewall as illustrated above for FIGS. 1 and 2. As illustrated, animal treatment pad 300 may include base 302. Base 302 may include padded area 304 to provide comfort to an animal utilizing embodiments described herein.

FIG. 4 is an illustrative schematic representation of a power control system 400 in accordance with embodiments of the present invention. A power control system, as contemplated by embodiments disclosed herein, provides a narrow spectrum light treatment at a comfortable temperature and includes an automated power conservation function. As illustrated, power source 402 provides sufficient electrical energy to provide the functions disclosed above. As may be appreciated, a power source may include without limitation, an alternating current power source, a direct current power source, and a solar cell power source. Where solar cell power sources are utilized, an energy storage device such as a battery may be required in some embodiments. Power source 402 provides power to the device through switch 404. As illustrated above, switch 404 may provide power conservation functions. As such, switches may include without limitation, a user activated switch, a pressure sensitive switch configured for automatically activating when an animal is located on the animal treatment pad, an inductive switch configured for automatically activating when an animal is detected on or near the animal treatment pad, and a motion switch configured for automatically activating when motion is detected on or near the animal treatment pad.

Further, as illustrated, narrow spectrum lamp 406 may be utilized to provide a narrow spectrum of light as well as heat for an animal. Narrow spectrum lamps may include without limitation, an incandescent bulb, a fluorescent bulb, and a plurality of light emitting diodes. In embodiments, narrow spectrum lamps have a wattage equivalent in a range of approximately 5 to 300 watts, more preferably 25 to 150 watts. In some embodiments, narrow spectrum lamps emit light in a spectrum range of approximately 520-435 nm. Intensity adjustment module 410 is provided to respond to input. In some embodiments, input may be a user adjustable input. That is, a user may manually set intensity to a desired level. In other embodiments, input may be provided by thermocouple 408. Thermocouple 408 may be configured to provide an ambient temperature input for intensity adjustment module 410. In this manner, a continuously comfortable and localized environment may be provided for pets utilizing embodiments disclosed herein. In order to achieve this environment, logic element 412 may be utilized to regulate or electronically drive intensity adjustment element 414 in response to an intensity curve corresponding with a thermocouple for example. Logic element embodiments may be enabled in any manner well known in the art without limitation without departing from the present invention. In some embodiments, logic element is hardware enabled, software enabled, or hardware and software enabled without limitation. In some embodiments, logic element may be in electronic communication with a computing device, which device may be utilized to provide user adjustable input. In some embodiments, a treatment regime may be logged with the computing device. In some embodiments, the intensity of the narrow spectrum lamp is adjustable to an intensity range of approximately 100 to 25% over a temperature range of approximately 20 to 100° F., more preferably to a temperature range of approximately 40 to 80° F.

In some embodiments, air circulating device 416 may be utilized to further control temperature. For example, in some embodiments, an air circulating device may be vented through opaque lamp shade (see FIG. 1, 116). In those embodiments, air circulating devices may be configured to both “push” and “pull” air through the shade. In this manner, heat from a lamp fixture may either be directed toward an animal (i.e. push) to provide additional heat for the animal or away from an animal (i.e. pull) to move heat away from the animal. Air circulating devices may be in electronic communication with logic described above to achieve a comfortable environment for an animal. In other embodiments, air circulating devices may be user adjustable. In still other embodiments, air circulating devices may be single speed, variable speed, or multi-speed.

In some embodiments, logic element may be further electronically coupled with a weighing device in order to track and store an animal's weight. As noted above, tracking an animal's weight may be particularly useful in determining whether a treatment regime is effective. For example, if weight loss increases with an increase in light exposure, a user may elect to reduce light exposure to stabilize weight or reverse weight loss. In another example, weight tracking may provide feedback to an owner to initiate an increase or decrease in amount of food being given to an animal or pet. Logic element may store weight associated data in any manner known in the art without departing from embodiments disclosed herein.

In some embodiments, logic element may be further electronically coupled with a timer in order to track exposure times. In an on-going a treatment regime, tracking exposure may provide data points for improving treatment. For example, if an adverse effect is noted, exposure times may be correlated with the effects to determine whether exposure should be reduced, or in some examples, increased. Likewise, if a beneficial effect is noted, exposure times may be similarly correlated. Logic element may store timer data in any manner known in the art without departing from embodiments disclosed herein.

FIG. 5 is an illustrative representation of user adjustable inputs 500 in accordance with embodiments of the present invention. It may be appreciated that the illustrated user adjustable inputs are provided for clarity in understanding embodiments of the present invention and should not be construed as limiting, with respect to shape, configuration or layout. Indeed, any number of configurations may be possible without departing from embodiments of the present invention. As illustrated, Tₘₐₓ input 502 may be utilized to adjust a maximum temperature at which a minimum light intensity is emitted. A Tₘᵢₙ input 504 may be utilized to adjust a minimum temperature at which a maximum light intensity is emitted. Likewise Iₘₐₓ input 506 may be utilized to adjust a maximum light intensity emitted at a minimum temperature. In addition, Iₘᵢₙ input 508 may be utilized to adjust a minimum light intensity emitted at a maximum temperature. In one embodiment power switch 510 may be utilized to power up or power down the device. Any power switch known in the art may be utilized without departing from the present invention. In other embodiments, power may be managed utilizing, without limitation, a pressure sensitive switch configured for automatically activating when an animal is on the pad, an inductive switch configured for automatically activating when an animal is detected, and a motion switch configured for automatically activating when motion is detected. A communication port 512 may be utilized to communicate with a computing device. Any manner of connection to communicate with a computing device known in the art may be utilized without departing from the present invention.

FIG. 6 is an illustrative graph 600 of an intensity curve for automatically adjusting light intensity in accordance with embodiments of the present invention. As noted above for FIG. 4, a logic element 412 may be in electronic communication with intensity adjustment element 414 and utilized to regulate or electronically drive intensity adjustment element 414. Any logic known in the art that is capable of providing functionality described herein may be utilized without departing from the present invention. In some embodiments, logic element is hardware enabled, software enabled, or hardware and software enabled without limitation. In some embodiments, logic element may be in electronic communication with a computing device, which device may be utilized to provide user adjustable input. As illustrated, intensity curve 606 adjusts intensity 602 generally downward over a range of temperatures 604. In embodiments, a maximum light intensity (Iₘₐₓ) 610 may be established either by default or in response to user input utilizing an adjustment input (see FIG. 5 above). As utilized herein, Iₘₐₓ corresponds with as maximum light intensity emitted at a minimum temperature. In embodiments, a minimum light intensity (Iₘᵢₙ) 612 may be established either by default or in response to user input utilizing an adjustment input (see FIG. 5 above). As utilized herein, Iₘᵢₙ corresponds with a minimum light intensity emitted at a maximum temperature. In embodiments, a minimum temperature (Tₘᵢₙ) 614 may be established either by default or in response to user input utilizing an adjustment input (see FIG. 5 above). As utilized herein, Tₘᵢₙ corresponds with a minimum temperature at which a maximum light intensity is emitted. In addition, in embodiments, a maximum temperature (Tₘₐₓ) 616 may be established either by default or in response to user input utilizing an adjustment input (see FIG. 5 above). As utilized herein, Tₘₐₓ corresponds with a maximum temperature at which a minimum light intensity is emitted. In some embodiments, logic may be in electronic communication with an air circulating device that is response to energy curve 606.

Utilizing user adjustment inputs, treatment may be tailored to a specific animal. Thus, for example as illustrated in FIG. 6, when temperatures are below 40° F. (i.e. Tₘᵢₙ 614), intensity remains at approximately 100% (i.e. Iₘₐₓ 610). Likewise as illustrated, when temperatures are above 80° F. (i.e. Tₘₐₓ 616), intensity remains at approximately 20% (i.e. Iₘᵢₙ 612). It is suggested that at intensities below 25% only nominal heat is produced. Thus, in some embodiments, an animal may benefit from exposure to narrow spectrum light but not be over heated when ambient temperatures are above a selected Tₘₐₓ. It may be appreciated that user adjustable inputs may be adjusted over any range of values. In one embodiment, default and user inputs may be adjustable to an intensity range of approximately 100 to 0% over a temperature range of approximately 0 to 100° F. As such, in some embodiments, heat generated from the use of a narrow spectrum lamp may provide warmth to an animal in addition to other benefits described herein. In addition, although intensity curve 606 is illustrated having a linear curve, any curve may be utilized without limitation and without departing from the present invention. For example, intensity curve 608 may be selected having a different curve adjustment which may be user selected in some embodiments.

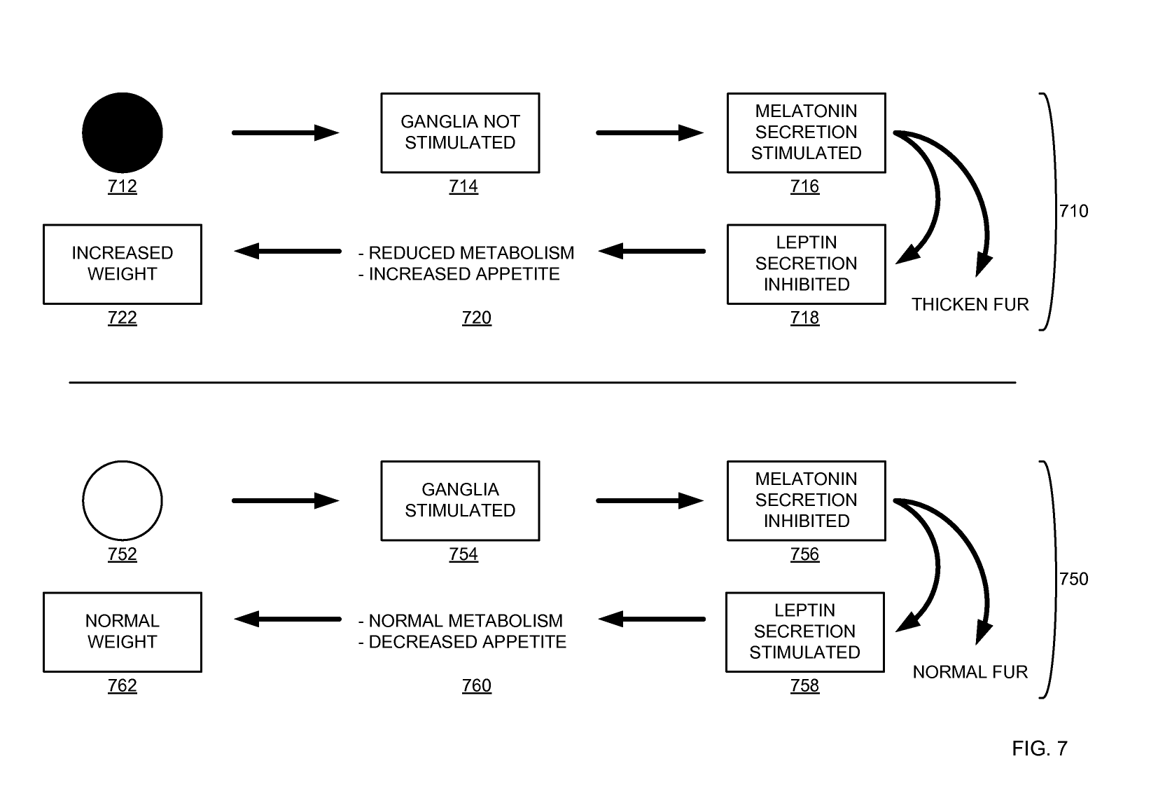

FIG. 7 is an illustrative representation of therapeutic effects in utilizing embodiments of the present invention. As noted above, and without being bound by theory, experimental use has demonstrated that use of embodiments provided herein have resulted in several beneficial aspects of pet care. For example, in utilizing embodiments provided herein, pets may be treated such that appetite increase during months preceding winter and during winter is avoided. It is suggested that the use of a narrow spectrum light may function to “trick” an animal's evolutionary control system to respond to what is essentially a continuous summer. As illustrated in FIG. 7, representations of a hibernation-like cycle 710 and an inhibited hibernation-like cycle 750 are exhibited. Although most pets do not hibernate, it is believed, without being bound by theory that hibernation mechanisms may operate similarly albeit to a lesser extent with pets.

In hibernation-like cycle 710, increased darkness 712 due to, for example, winter solar patterns, may result in decreased nerve stimulation in an animal's retinal ganglia. One result of the decreased nerve stimulation is that melanopsin production is inhibited 714. Melanopsin is a photo pigment found in specialized photosensitive ganglion cells of the retina that are involved in the regulation of circadian rhythms, pupillary light reflex, and other non-visual responses to light. In response to a lack of melanopsin, the pineal gland of some mammals may be stimulated to secrete melatonin 716 which may thicken fur and inhibit leptin secretion 718. Leptin appears to work as a feedback mechanism to signal the body regarding the amount of body fat and its distribution. Decreased leptin increases appetite and increases the body's ability to lay down fat. In contrast, higher levels of leptin decreases appetite and decrease the body's ability to lay down fat. Thus, decreased leptin may result in any of a number of physiological changes including, but not limited to: reduced metabolism and increased appetite 720. Without being bound by theory, a net result may be increased weight during winter months 722.

In contrast, in an inhibited hibernation-like cycle 750, embodiments utilizing specific wavelengths of light 752 may result in nerve stimulation in an animal's retinal ganglia. One result of the nerve stimulation is that melanopsin production is stimulated 754. As noted above, melanopsin is a photo pigment found in specialized photosensitive ganglion cells of the retina that are involved in the regulation of circadian rhythms, pupillary light reflex, and other non-visual responses to light. In response to melanopsin production, the pineal gland of some mammals may inhibit melatonin secretion 756 which may, in turn, stimulate leptin secretion 758. As noted above, leptin appears to work as a feedback mechanism to signal the body regarding the amount of body fat and its distribution. Decreased leptin increases appetite and increases the body's ability to lay down fat. In contrast, higher levels of leptin decreases appetite and decrease the body's ability to lay down fat. Thus, leptin secretion may result in any of a number of physiological changes including, but not limited to: normal metabolism and decreased appetite 760. Without being bound by theory, a net result may be normal weight during treatment periods 762.

While this invention has been described in terms of several embodiments, there are alterations, permutations, and equivalents, which fall within the scope of this invention. It should also be noted that there are many alternative ways of implementing the methods and apparatuses of the present invention. Furthermore, unless explicitly stated, any method embodiments described herein are not constrained to a particular order or sequence. Further, the Abstract is provided herein for convenience and should not be employed to construe or limit the overall invention, which is expressed in the claims. It is therefore intended that the following appended claims be interpreted as including all such alterations, permutations, and equivalents as fall within the true spirit and scope of the present invention.

What is claimed is:

1. A method of weight treatment in an animal comprising:
stimulating a retinal ganglia of the animal by an exposure to a narrow spectrum light.

2. The method of claim 1, wherein the stimulating the retinal ganglia of the animal is associated with a stimulation of melanopsin.

3. The method of claim 2, wherein the stimulation of melanopsin is associated with a inhibition of melatonin secretion.

4. The method of claim 3, wherein the inhibition of melatonin secretion is associated with a stimulation of leptin secretion.

5. The method of claim 4, wherein the stimulation of leptin secretion is associated with a decrease in appetite in the animal.

6. The method of claim 1, wherein the exposure to the narrow spectrum light is associated with a weight loss in the animal.

7. The method of claim 1, wherein the narrow spectrum light is in a spectrum range of approximately 435 to 520 nanometers.

8. The method of claim 1, wherein the exposure to the narrow spectrum light is continuous.

9. The method of claim 1, wherein the exposure to the narrow spectrum light is associated with an inhibited hibernation-like cycle in the animal.

10. The method of claim 1, wherein the narrow spectrum light is provided by a source selected from the group consisting of: an incandescent bulb, a fluorescent bulb, and a plurality of light emitting diodes, and wherein the lamp has a wattage in a range of approximately 5 to 300 watts.
